(19) United States
(12) Patent Application Publication
(10) Pub. No.: US 2023/0193187 A1
(43) Pub. Date: Jun. 22, 2023

(54) SYSTEM FOR DETERMINING MULTINUCLEATED STATE OF MYOBLAST CELLS

(71) Applicant: TERUMO KABUSHIKI KAISHA, Tokyo (JP)

(72) Inventor: Eri NOGUCHI, Ashigarakami-gun (JP)

(73) Assignee: TERUMO KABUSHIKI KAISHA, Tokyo (JP)

(21) Appl. No.: 18/165,848

(22) Filed: Feb. 7, 2023

Related U.S. Application Data
(63) Continuation of application No. PCT/JP2021/036055, filed on Sep. 30, 2021.

(30) Foreign Application Priority Data
Sep. 30, 2020 (JP) 2020-165844

(51) Int. Cl. C12M 1/34; C12M 1/00; G01N 1/30; G01N 15/14

(52) U.S. Cl. CPC C12M 41/46; C12M 41/14; G01N 1/30; G01N 15/1468

(57) ABSTRACT

A stable and efficient system that determines a multinucleated state of myoblast cells, the system including a storage unit that stores a cell culture substrate containing the myoblast cells, a measurement unit that measures a shape of the myoblast cells, and an analysis unit that calculates the number of nuclei in the individual myoblast cells based on a parameter.

### CROSS REFERENCES TO RELATED APPLICATIONS

This application is a continuation of International Patent Application No. PCT/JP2021/036055 filed on Sep. 30, 2021, which claims priority to Japanese Patent Application No. 2020-165844 filed on Sep. 30, 2020, the entire content of both of which is incorporated herein by reference.

### TECHNOLOGICAL FIELD

The present disclosure generally relates to a system and the like that determines a multinucleated state of myoblast cells.

### BACKGROUND DISCUSSION

In recent years, attempts have been made to transplant various cells for repair of damaged tissues and the like. For example, attempts have been made to use fetal cardiomyocytes, myoblast cells, mesenchymal stem cells, cardiac stem cells, ES cells, iPS cells, and the like for repair of myocardial tissue damaged by ischemic heart disease such as angina pectoris and myocardial infarction (Haraguchi Y. et al., Stem Cells Transl. Med., 1(2), 136-141 (2012)).

As a part of such attempts, a cell structure formed using a scaffold and a sheet-shaped cell culture in which cells are formed in a sheet shape have been developed (Sawa Y. et al., Surg. Today, 42(2), 181-184 (2012)).

The sheet-shaped cell culture is extremely useful for the repair of damaged tissues and the like because the sheet-shaped cell culture can fix a large amount of desired cells to a damaged site and can transplant a moderately organized cell population in accordance with the characteristics of the recipient tissue.

The sheet-shaped cell culture is prepared by culturing desired cells on a surface of a cell culture substrate, and peeling off a sheet-shaped culture obtained by forming a layer structure of cells from the cell culture substrate so as not to destroy the structure.

In recent years, it is known that the quality of a sheet-shaped cell culture may vary depending on the properties of cells constituting the sheet-shaped cell culture. For example, it has been reported that in a sheet-shaped cell culture of myoblast cells, production of cytokines promoting myocardial regeneration is higher in a group in which myoblast cells are dissociated, and more multinucleated cells are formed when the dissociated cells are incubated in a medium (WO2012/023562). In this literature, it is reported that an evaluation method capable of providing a high-quality sheet-shaped cell culture has been developed based on such findings.

### SUMMARY

Disclosed here is a stable and efficient unit that determines a multinucleated state of myoblast cells.

The present inventor has studied a method for preventing variation in quality of a sheet-shaped cell culture, and has considered that when determining a multinucleated state of myoblast cells, it is forced to rely on visual determination of stained cells by an operator, and a result of the visual determination varies among operators, which may cause variation in determination of the multinucleated state of myoblast cells. Then, as a result of intensive studies to provide a stable and efficient unit that determines the multinucleated state of myoblast cells, it has been found that the multinucleated state of myoblast cells can be determined by measuring a shape of myoblast cells and calculating the number of nuclei in individual myoblast cells based on a parameter, and as a result of further studies based on such findings, the present disclosure has been completed.

That is, the present disclosure relates to the following.

A system that determines a multinucleated state of myoblast cells, the system including: a storage unit that stores a cell culture substrate including the myoblast cells; a measurement unit that measures a parameter of the myoblast cells; and an analysis unit that calculates a number of nuclei in the individual myoblast cells based on the parameter.

The system according to [1], in which the parameter of the myoblast cells includes shapes of outlines of the myoblast cells and the nuclei.

The system according to [1] or [2], in which the measurement unit includes an imaging unit.

The system according to [2] or [3], in which the parameter is a shape and/or a position of an outline of the myoblast cells, a shape and/or a position of an outline of the nuclei, and/or a distance between an outline of the myoblast cells or the nuclei and an outline of the myoblast cells or the nuclei.

[5] The system according to [3] or [4], in which the analysis unit distinguishes shapes of outlines of the myoblast cells and the nuclei in an image data from the imaging unit, and calculates the number of nuclei in the individual myoblast cells based on the parameter.

[6] The system according to any of [1] to [5], in which the myoblast cells and/or the nuclei are unstained.

[7] The system according to any one of [1] to [6], further including a learning unit that extracts excess or deficiency of the parameter based on information from the analysis unit.

[8] The system according to [7], further including an update unit that updates the parameter based on the excess or deficiency of the parameter extracted by the learning unit.

[9] A method of determining a multinucleated state of myoblast cells includes: providing a cell culture substrate including the myoblast cells, measuring a parameter of the myoblast cells, and calculating a number of nuclei in the individual myoblast cells based on the parameter.

According to the present disclosure, a stable and efficient unit that determines a multinucleated state of myoblast cells can be provided. In particular, according to the present disclosure, by measuring the shape of the myoblast cells and calculating the number of nuclei in the individual myoblast cells on the basis of the parameter, it is possible to systematize the determination of the multinucleated state, which conventionally had to be entrusted to visual determination of stained cells by an operator, and a result of the visual determination may vary between operators, and it is possible to realize stabilization and efficiency of work. Furthermore, according to the present disclosure, the process of cell staining and cell observation after conventional cell culture can be omitted, and the multinucleated state of the myoblast cells can be directly determined during cell culture. From the above, the disclosure here makes it possible to realize excellent quality control of a sheet-shaped cell culture by such systemization and to stably and efficiently supply a high-quality sheet-shaped cell culture.

### BRIEF DESCRIPTION OF THE DRAWINGS

FIG. 1 illustrates a flow diagram of an exemplary embodiment of a system that determines the multinucleated state of individual cells.

FIG. 2 illustrates an example of components of a system that determines a multinucleated state of myoblast cells.

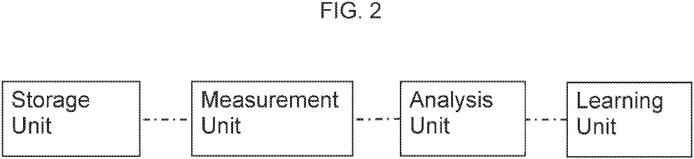

### DETAILED DESCRIPTION

Unless otherwise defined herein, all technical and scientific terms used herein have the same meaning as commonly understood by one of ordinary skill in the art. All patents, applications and other publications and information referenced herein are hereby incorporated by reference in their entirety.

### System That Determines Multinucleated State of Myoblast Cells

One aspect of the present disclosure relates to a system that determines a multinucleated state of myoblast cells (which may be referred to as “the system of the present disclosure”), the system including a storage unit that stores a cell culture substrate containing the myoblast cells, a measurement unit that measures a shape of the myoblast cells, and an analysis unit that calculates a number of nuclei in the individual myoblast cells based on a parameter. The system of the present disclosure may further include a learning unit that extracts excess or deficiency of the parameter on the basis of information from the analysis unit. The system of the present disclosure may further include an update unit that updates the parameter based on the excess or deficiency of the parameter extracted by the learning unit.

In the present disclosure, the “myoblast cells” refers to any myoblasts separated from a skeletal muscle tissue. The myoblast cells are mononuclear cells from which muscle fibers are derived. The myoblast cells fuse to each other and differentiate into multinucleated cells (or myotubes) to form muscle fibers. The fusion of the myoblast cells may be between allogeneic cells or between xenogeneic cells. In the present disclosure, the myoblast cells may include, for convenience, a multinucleated cell formed by cell fusion of the cells.

A skeletal muscle tissue may be derived from any organism. Such organisms include, but are not limited to, humans, non-human primates, rodents (mouse, rat, hamster, guinea pig, etc.), dogs, cats, pigs, horses, cows, goats, sheep, and the like. In a case where the cells separated from the skeletal muscle tissue are used for transplantation, the skeletal muscle tissue used in the present disclosure can avoid rejection by using autologous cells separated using the skeletal muscle tissue collected from a transplantation subject (recipient) itself. However, it is also possible to use heterologous cells or homologous non-autologous cells separated using heterologous or homologous non-autologous skeletal muscle tissue.

In the present disclosure, the “cell culture substrate” is not particularly limited as long as myoblast cells can form a cell culture thereon, and includes, for example, containers of various materials and/or shapes, solid or semi-solid surfaces in the containers, and the like. The container is preferably a structure or material that does not allow permeation of a liquid such as a culture solution. Examples of such a material include, but are not limited to, polyethylene, polypropylene, Teflon®, polyethylene terephthalate, polymethyl methacrylate, nylon 6,6, polyvinyl alcohol, cellulose, silicon, polystyrene, glass, polyacrylamide, polydimethylacrylamide, and metals (for example, iron, stainless steel, aluminum, copper, brass). Furthermore, it is also preferred that the container has at least one flat surface. Examples of such a container include, but are not limited to, a culture container having a bottom surface composed of a cell culture substrate capable of forming a cell culture and a liquid-impermeable side surface. Specific examples of such a culture container include, but are not limited to, a cell culture dish, a cell culture bottle, and the like. The bottom surface of the container may be transparent or opaque. When the bottom surface of the container is transparent, cells can be observed, counted, and the like from a back side of the container. Furthermore, the container may also have a solid or semi-solid surface therein. Examples of the solid surface include plates and containers of various materials as described above, and examples of the semi-solid surface include a gel and a soft polymer matrix. The cell culture substrate may be produced using the above materials, or a commercially available material may be used.

In the present disclosure, the “storage unit” may be any unit as long as it can store the cell culture substrate containing myoblast cells, and for example, may have a control unit capable of controlling the surrounding environment of the cell culture substrate. Examples of the surrounding environment include, but are not limited to, temperature, pressure, humidity, CO₂ concentration, and the like. Therefore, the control unit may include an input unit that inputs a setting to be controlled, a recording unit that records a setting value, a measurement unit that measures the current state of the surrounding environment, an output unit that outputs information measured by the measurement unit, and the like. Furthermore, such a control unit may share a CPU, the input unit, the output unit and/or the recording unit with the analysis unit. That is, a CPU may serve as the control unit, the input unit, the output unit the recording unit and/or the analysis unit, with such CPU operating as one or more of those units s described herein.

In one aspect, the storage unit can keep the surroundings of the cell culture substrate in an environment suitable for cell culture. Therefore, the storage unit in such an aspect can function as a cell culture incubator. In this aspect, it is possible to culture cells using the storage unit as an incubator and determine a multinucleated state of myoblast cells in parallel with incubation.

In the present disclosure, the “shape of myoblast cells” may be any shape that captures structural features such as cells, cell membranes, cytoplasm, organelles, and nuclei, but includes, for example, shapes of outlines of myoblast cells and nuclei. The myoblast cells and/or nuclei may be unstained or stained.

In the present disclosure, the “measurement unit” may be any unit as long as it can measure a shape of myoblast cells, and includes, for example, an imaging device, a transmitted light detector, a hue color difference meter, and the like. From the viewpoint of versatility of data, diversity of processing, and the like, it is preferable that the measurement unit includes an imaging unit, and the shape of myoblast cells is acquired as image data.

In the present disclosure, the “imaging unit” may be any unit as long as it can image a shape of myoblast cells, and includes, for example, an optical microscope equipped with a camera. The optical microscope may be any microscope as long as it is suitable for imaging the shape of myoblast cells, for example, a bright field microscope, a dark field microscope, a phase contrast microscope, a differential interference contrast microscope, a polarization microscope, a fluorescence microscope, a confocal laser microscope, a total reflection illumination fluorescence microscope, a Raman microscope, or the like. From the viewpoint of versatility of data, diversity of processing, and the like, the camera is preferably a digital camera, and the shape of myoblast cells is preferably acquired as image data.

In the present disclosure, the “parameter” may be any parameter that captures structural characteristics such as cells, cell membranes, cytoplasm, organelles, and nuclei, and is, for example, a shape and/or a position of an outline of myoblast cells, a shape and/or a position of an outline of nuclei, and/or a distance between an outline of myoblast cells or nuclei and an outline of cells or nuclei.

In the present disclosure, the “analysis unit” may be any unit as long as the number of nuclei in individual myoblast cells can be calculated on the basis of a parameter, and includes, for example, image analysis software operating on a computer. The analysis performed by the analysis unit can vary depending on a format of the obtained data. Although not limited thereto, for example, the shape of myoblast cells may be obtained by processing the obtained image data, or the shape of myoblast cells in one view may be counted in the obtained image data.

The analysis unit may distinguish the shapes of the outline of the myoblast cells and the nuclei in the image data from the imaging unit, and calculate the number of nuclei in the individual myoblast cells based on the parameter.

The shape of the myoblast cells can be recorded in a recording medium in the analysis unit before being analyzed by the analysis unit. Therefore, the analysis unit may include a recording unit. The recording unit may be electrically recorded, for example, a magnetic tape, a magnetic disk, an optical disk, a magneto-optical disk, a flash memory, or the like, or may be physically recorded, for example, paper, a photograph, or the like.

The system of the present disclosure calculates the number of nuclei in the individual myoblast cells based on the parameter, and determines that the myoblast cells have become multinucleated (differentiated into myotubes) in a case where the number of nuclei in the individual myoblast cells is two or more.

The analysis unit can also calculate a change rate of the multinuclear state from a temporal change of the multinuclear state. For example, the change rate of the multinucleated state can be calculated by (the number of nuclei per cell after a predetermined time elapses from a certain time point)/(the number of nuclei per cell at a certain time point). The number of nuclei per cell can be calculated for one or more cells and also for one or more cell populations. In a case where the number of nuclei per cell is calculated for two or more cells, an average value and standard deviation thereof can be calculated. Here, for example, a predetermined threshold value such as 2, 3, 4, 5, 6, or the like is set, and in a case where the change rate of the multinucleated state is more than or equal to the threshold value, it can be determined that the entire myoblast cells have become multinucleated. Thereby, the multi-nucleating ability of the myoblast cells can also be determined. For example, in a case where the change rate is larger than 1, it can be determined that the multi-nucleating ability of the myoblast cells is recognized, and in a case where the change rate is smaller than 1, it can be determined that the multi-nucleating ability of the myoblast cells is not recognized.

A setting value of the threshold value may change depending on the number of myoblast cells to be seeded and the like, and may be an arbitrary value input according to the form of use, or may be a value recorded in the recording unit in advance. Therefore, the analysis unit may include an input unit. As a form of the input unit, for example, any input unit known to those skilled in the art, such as a keyboard, a sensor (the sensor may be the same as a measurement device of the measurement unit), and a touch panel, can be used. Since the setting value changes depending on a value measured by the measurement unit, the setting value can be arbitrarily input at the time of use. An input unit of the setting value may be common to or different from the above-described input unit.

In the analysis unit, preferably, the shape of the myoblast cells measured by the measurement unit is output to the outside as information. Therefore, the analysis unit may include an output unit of the information. The output unit of the information may be, for example, an external monitor, a lamp, a buzzer, transmission to a mobile phone or an Internet mail address registered in advance, or the like to notify a person who operates information on completion of peeling, or may be output to a recording unit such as a printer.

In the present disclosure, the “multinucleated state of myoblast cells” refers to a state in which two or more nuclei are present in one myoblast cell. Such a state may be formed by fusion of two or more myoblast cells.

In the present disclosure, the “learning unit” is a unit that extracts excess or deficiency of a parameter on the basis of information from the analysis unit. The learning unit extracts excess or deficiency of the parameter by associating the parameter of the analysis unit with the number of nuclei in individual myoblast cells calculated by the analysis unit. For example, if the determination of the multinucleated state of myoblast cells is not appropriately made and even mononuclear cells are determined to be in the multinucleated state, the learning unit can determine that excess or deficiency of the parameter (for example, the shape and/or position of the outline of myoblast cells, the shape and/or position of the outline of nuclei, and/or the distance between the outline of myoblast cells or nuclei and the outline of myoblast cells or nuclei) occurs. Note that whether or not the determination has been appropriately made can be determined by cell staining and cell observation for the same cell.

The learning unit may further include a learning storage unit. The learning storage unit is a unit that accumulates and stores a parameter of the analysis unit and information from the analysis unit in the parameter, and includes various electronic storage media similarly to the storage unit. The learning unit may extract excess or deficiency of the parameter with higher accuracy with reference to the data stored in the learning storage unit. Furthermore, the learning unit may further include a learning input unit and a learning output unit. The learning input unit is a unit where an operator of the system of the present disclosure, another unit of the system, or another system inputs information that can enable more efficient and highly accurate learning as necessary, and the learning output unit is a unit that emits a predetermined signal on the basis of the extracted excess or deficiency of the parameter. The learning input unit and the learning output unit may include interfaces similar to the input unit and the output unit, respectively.

In the present invention, the “update unit” is a unit that updates a parameter on the basis of excess or deficiency of the parameter extracted by the learning unit. The update unit receives information from the learning unit, creates a new parameter reflecting the information, and updates the parameter of the analysis unit with the created parameter. The update unit may include an output interface for outputting to the analysis unit, or may be integrated with the analysis unit. Alternatively, the update unit is integrated with the learning unit, and may not receive the information from the learning unit, but may create a new parameter and update the parameter of the analysis unit on the basis of the information from the analysis unit.

### Method of Determining Multinucleated State of Myoblast Cells

Another aspect of the present disclosure relates to a method of determining a multinucleated state of myoblast cells (which may be referred to as “the method of the present disclosure”), including the steps of: providing a cell culture substrate including the myoblast cells, measuring a shape of the myoblast cells, and calculating the number of nuclei in the individual myoblast cells based on a parameter.

The method of the present disclosure may further include a step of determining that the cell is multinucleated in a case where the number of nuclei in the individual myoblast cells is two or more.

Hereinafter, the system of the present disclosure will be described in more detail with reference to the drawings, which show exemplary embodiments that are in accordance with the present disclosure. The present disclosure is not limited to these exemplary embodiments.

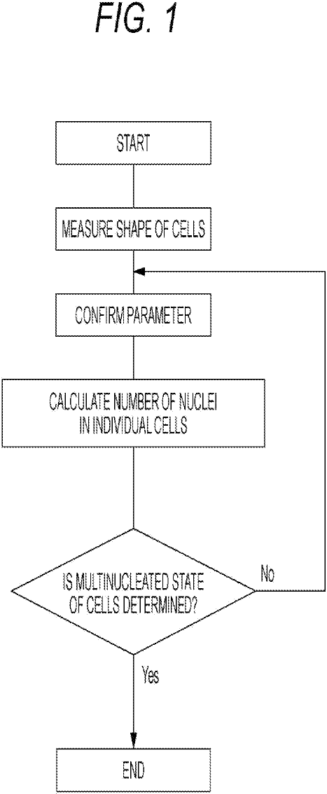

FIG. 1 illustrates a flow diagram of processing of the system of the present disclosure in one aspect. In the system of the present disclosure, when a signal is input, the measurement unit starts measurement of a shape of myoblast cells contained in the cell culture substrate stored in the storage unit. The system of the present disclosure then confirms and sets a parameter for analyzing the measured shape of the myoblast cells. The system of the present disclosure calculates the number of nuclei in the individual myoblast cells based on the parameter, and determines that the myoblast cells are multinucleated in a case where the number of nuclei in the individual myoblast cells is two or more. In a case where the determination has been made appropriately, the system of the present disclosure can output a signal and terminate the flow. Determination whether or not the determination has been made appropriately can be made by cell staining and cell observation for the same cell.

In one aspect, the system of the present disclosure determines whether or not an object to be measured is suitable for determining a multinucleated state of the myoblast cells prior to initiating the measurement of the shape of the myoblast cells.

For example, the system of the present disclosure determines whether or not cells are adsorbed or adhered to the cell culture substrate prior to initiating the measurement of the shape of the myoblast cells. In a case where the cells are not adsorbed or adhered to the cell culture substrate and the cells are detached from the cell culture substrate, the system of the present disclosure can output a signal to terminate the flow completely or temporarily.

Furthermore, for example, the system of the present disclosure determines whether or not cells are excessively adsorbed or adhered to the cell culture substrate before starting the measurement of the shape of the myoblast cells. In a case where the excessive adsorption or adhesion of cells to the cell culture substrate may result in densification, lamination, etc. of the cells on the cell culture substrate, making it difficult to recognize the individual cells, the system of the present disclosure can output a signal to completely or temporarily terminate the flow.

Furthermore, for example, the system of the present disclosure determines whether or not contaminants such as solids other than cells are mixed in the cell culture substrate before starting the measurement of the shape of the myoblast cells. In a case where the cell culture substrate is contaminated with contaminants such as solids other than cells, the system of the present disclosure can output a signal to terminate the flow completely or temporarily.

Furthermore, in a case where stained cells are to be measured, for example, the system of the present disclosure determines whether or not the cells on the cell culture substrate are appropriately stained before starting measurement of the shape of the myoblast cells. Such determination can be made, for example, from the viewpoint of thin dyeing, lack of uniformity of dyeing, precipitation of components of a dyeing solution, contamination of foreign matters at the time of dyeing, detachment of cells due to physical factors during dyeing, and the like. In a case where the cells are not appropriately stained, the system of the present disclosure can output a signal to terminate the flow completely or temporarily.

The system of the present disclosure can determine that the myoblast cells are multinucleated in a case where the shape of the outline of the myoblast cells is an elongated shape (elongated spindle shape, elliptical shape, rectangular shape, linear shape, string shape, approximate shape thereof, and the like) and two or more shapes of the outlines of the nuclei fall within the same shape. At this time, the shapes of the outlines of the two or more nuclei contained in the same shape may be in a case where there is a certain directionality in the arrangement, or in a case where there is no such a certain directionality and the shapes of the outlines of the two or more nuclei are simply aggregated, and in either case, it can be determined that the myoblast cells are multinucleated. Here, the “certain directionality” refers to that two or more nuclei are arranged in contact with and/or spaced apart from each other in a longitudinal direction of an elongated shape of the an outline of the myoblast cells (elongated spindle shape, elliptical shape, rectangular shape, linear shape, string shape, approximate shape thereof, and the like).

In contrast, the system of the present disclosure may determine that myoblast cells are not multinucleated in a case where the shape of the outline of the myoblast cells is not an elongated shape (elongated spindle shape, elliptical shape, rectangular shape, linear shape, string shape, approximate shape thereof, and the like) and/or in a case where there are not two or more shapes of the outlines of the nuclei within the same shape. Furthermore, the system of the present disclosure may also determine that the myoblast cells are not multinucleated when the shapes of outlines of the myoblast cells and/or nuclei overlap and may at one glance be recognized as multinucleated cells.

Furthermore, the system of the present disclosure can determine that the myoblast cells are multinucleated even in a case where the shape of the outline of the myoblast cells is an elongated shape (elongated spindle shape, elliptical shape, rectangular shape, linear shape, string shape, approximate shape thereof, and the like), two or more shapes of the outlines of the nuclei are contained in the same shape, and the shapes of the outlines of two or more nuclei are aggregated.

Moreover, the system of the present disclosure can also determine that the myoblast cells are multinucleated in a case where the shape of the outline of the myoblast cells is an elongated shape (elongated spindle shape, elliptical shape, rectangular shape, linear shape, string shape, approximate shape thereof, and the like), two or more shapes of the outlines of the nuclei are contained in the same shape, the shapes of the outlines of two or more nuclei are aggregated, and two or more aggregates are contained in the same shape.

In a case where the determination has been made appropriately, the system of the present disclosure can output a signal and terminate the flow. Determination whether or not the determination has been made appropriately can be made by cell staining and cell observation for the same cell.

In a case where the determination has not been made appropriately, the system of the present disclosure outputs a signal to confirm and set a parameter for analyzing the measured shape of the myoblast cells again. The system of the present disclosure then calculates the number of nuclei in the individual myoblast cells based on the parameter and determines that the myoblast cells have been multinucleated in a case where the number of nuclei in the individual myoblast cells is greater than or equal to 2. In a case where the determination has been made appropriately, the system of the present disclosure can output a signal and terminate the flow. In a case where the determination is not appropriately made, the above flow is repeated again.

The system of the present disclosure may further include the learning unit and the update unit. A signal may be output from the analysis unit to the learning unit, and in these units, the parameter may be changed and applied (updated) to the analysis unit. The learning unit may extract excess or deficiency of the parameter on the basis of the information from the analysis unit. The update unit may update the parameter based on the excess or deficiency of the parameter extracted by the learning unit.

In one aspect, the system of the present disclosure can determine the multinucleated state of the myoblast cells over time. In the system of the present disclosure, when a signal is input, the measurement unit starts measurement of a shape of myoblast cells contained in the cell culture substrate stored in the storage unit. The system of the present disclosure then confirms and sets a parameter for analyzing the measured shape of the myoblast cells. The system of the present disclosure calculates the number of nuclei in the individual myoblast cells based on the parameter, and determines that the myoblast cells are multinucleated in a case where the number of nuclei in the individual myoblast cells is two or more. After the lapse of a predetermined time, the system of the present disclosure starts again the measurement of the shape of the myoblast cells contained in the cell culture substrate stored in the storage unit by the measurement unit, and repeats the above flow.

The analysis unit can also calculate a change rate of the multinuclear state from a temporal change of the multinuclear state. For example, the change rate of the multinucleated state can be calculated by (the number of nuclei per cell after a predetermined time elapses from a certain time point)/(the number of nuclei per cell at a certain time point). The number of nuclei per cell can be calculated for one or more cells and also for one or more cell populations. In a case where the number of nuclei per cell is calculated for two or more cells, an average value and standard deviation thereof can be calculated. Here, for example, a predetermined threshold value such as 2, 3, 4, 5, 6, or the like is set, and in a case where the change rate of the multinucleated state is more than or equal to the threshold value, it can be determined that the entire myoblast cells have become multinucleated. Thereby, the multi-nucleating ability of the myoblast cells can also be determined. For example, in a case where the change rate is larger than 1, it can be determined that the multi-nucleating ability of the myoblast cells is recognized, and in a case where the change rate is smaller than 1, it can be determined that the multi-nucleating ability of the myoblast cells is not recognized.

In one aspect, in the system of the present disclosure, when a signal is input, the imaging unit starts to measure the shape of the outline of the myoblast cells and nuclei contained in the cell culture substrate stored in the storage unit. The system of the present disclosure then confirms and sets a parameter (a shape and/or a position of an outline of myoblast cells, a shape and/or a position of an outline of nuclei and/or a distance between an outline of myoblast cells or nuclei and an outline of myoblast cells or nuclei) for analyzing the measured shape of the outline of the myoblast cells and nuclei. The system of the present disclosure calculates the number of nuclei in the individual myoblast cells based on the parameter, and determines that the myoblast cells are multinucleated in a case where the number of nuclei in the individual myoblast cells is two or more. In a case where the determination has been made appropriately, the system of the present disclosure can output a signal and terminate the flow. Determination whether or not the determination has been made appropriately can be made by cell staining and cell observation on the same myoblast cells.

The analysis unit may distinguish the shapes of the outline of the myoblast cells and the nuclei in the image data from the imaging unit, and calculate the number of nuclei in the individual myoblast cells based on the parameter. The myoblast cells and/or nuclei may be unstained.

According to the system of the present disclosure described above, since the presence of the myoblast cells having a multi-nucleating ability in a cell population and the proportion thereof can be confirmed, the quality of a cell culture (for example, a sheet-shaped cell culture including myoblast cells) prepared by culturing the cell population can be stably and efficiently managed.

Although one aspect of the system of the present disclosure has been described above, it should be understood that various aspects other than the above are possible. Therefore, various aspects obtained by modifying the above aspects without departing from the spirit of the present disclosure are also included in the scope of the present disclosure, and such modifications are understandable to those skilled in the art.

The components constituting the system of the present disclosure can be arranged in various manners as long as the predetermined object can be achieved, and can be combined and integrated as necessary.

What is claimed is:

1. A method of determining a multinucleated state of individual myoblast cells, the method comprising:
providing a cell culture substrate that includes the individual myoblast cells;
measuring a parameter of the individual myoblast cells in the cell culture substrate; and
calculating a number of nuclei in the individual myoblast cells based on the parameter.

2. The method of claim 1, further comprising determining that the individual myoblast cells are multinucleated when the number of the nuclei in the individual myoblast cells is two or more.

3. A system that determines a multinucleated state of individual myoblast cells, the system comprising:
a storage unit that stores a cell culture substrate including the individual myoblast cells;
a measurement unit that measures a parameter of the individual myoblast cells; and
an analysis unit that calculates a number of nuclei in the individual myoblast cells based on the parameter.

4. The system according to claim 3, wherein the parameter of the individual myoblast cells includes shapes of outlines of the myoblast cells and the nuclei.

5. The system according to claim 3, wherein the measurement unit includes an imaging unit.

6. The system according to claim 3, wherein the parameter is a shape and/or a position of an outline of the individual myoblast cells, a shape and/or a position of an outline of the nuclei, and/or a distance between an outline of the individual myoblast cells or the nuclei and an outline of the individual myoblast cells or the nuclei.

7. The system according to claim 5, wherein the analysis unit distinguishes shapes of outlines of the individual myoblast cells and the nuclei in an image data from the imaging unit, and calculates the number of nuclei in the individual myoblast cells based on the parameter.

8. The system according to claim 3, wherein the individual myoblast cells and/or the nuclei are unstained.

9. The system according to claim 3, wherein the individual myoblast cells are derived from a skeletal muscle tissue of a human.

10. The system according to claim 3, wherein the cell culture substrate is a container made from a material selected from the group consisting of polyethylene, polypropylene, polyethylene terephthalate, polymethyl methacrylate, nylon 6,6, polyvinyl alcohol, cellulose, silicon, polystyrene, glass, polyacrylamide, polydimethylacrylamide and metals.

11. The system according to claim 5, wherein the imaging unit includes an optical microscope equipped with a camera.

12. The system according to claim 3, wherein the storage unit is a cell culture incubator.

13. The system according to claim 3, wherein the analysis unit includes one of a magnetic tape, a magnetic disk, an optical disk, a magneto-optical disk and a flash memory.
